(19) United States
(12) Patent Application Publication
(10) Pub. No.: US 2002/0165360 A1
(43) Pub. Date: Nov. 7, 2002

(54) Chimeric effector cell receptors against carcinoembryonic antigen

(76) Inventor: Richard P. Junghans, Boston, MA (US)

Correspondence Address: Richard P. Junghans, Ph.D., M.D., One Lyndeboro Place, Boston, MA, 02116, US

(21) Appl. No.: 10/006,771

(22) Filed: Dec. 10, 2001

Related U.S. Application Data
(60) Provisional application No. 60/250,090, filed on Nov. 30, 2000.
(60) Provisional application No. 60/250,087, filed on Nov. 30, 2000.

(51) Int. Cl. C07K 16/46

(52) U.S. Cl. 530/387.3; 530/391.1

(57) ABSTRACT

This invention relates to a specific humanized antibody against carcinoembryonic antigen (CEA) called hMN14 when prepared as a chimeric molecule with signaling molecules of T cells and other effector cells, and the use thereof in the treatment of cancers expressing CEA.

### RELATED APPLICATIONS

This application claims priority from US Provisional Patent Application No. 60/250,090, filed on Nov. 30, 2000, the contents of which are hereby incorporated by reference. This application also is related to U.S. Provisional Patent Application No. 60/250,087, “Method to improve protein expression by removal of cysteines,” filed on Nov. 30, 2000.

### REFERENCES CITED

U.S. Patent Documents:

U.S. Pat. No. 5,874,540 Hansen et al., 1994

U.S. Pat. No. 6,319,494 Capon et al., 1995

Other References:

Brinkmann et al, 1993, Proc Natl Acad Sci USA 90:7538-42.

Moritz et al, 1995, Gene Therapy 2:539-46.

Nolan et al, 1999, Clin Cancer Res 5:3928-41.

### FIELD OF THE INVENTION

The invention relates to immuno-gene therapy of CEA-expressing cancers.

### STATEMENT ON FEDERALLY-SPONSORED R&D

No federal funds were used in the creation of this invention.

### BACKGROUND OF THE INVENTION

The tumor-associated marker, carcinoembryonic antigen (CEA) is expressed on many tumors of epithelial origin—colorectal, breast, lung and others—and it has a profile of expression in normal tissues that will plausibly allow selective targeting of tumorous expression of the antigen. Nearly 150,000 Americans die each year from CEA-expressing cancers. T cells can penetrate virtually every biologic space and have the power to dispose of normal or malignant cells as seen in viral and autoimmune diseases and in the rare spontaneous remissions of cancer. However, T cells are readily tolerant to self or tumor antigens, and “immune surveillance” has manifestly failed in every cancer that is clinically apparent. There is a strong need and value for means to direct T cells against CEA-expressing cancers.

### BRIEF SUMMARY OF THE INVENTION

The humanized antibody against CEA has been prepared called hMN14 (Hansen et al, 1994). It is the goal of this patent to supply the specificities and affinities to patient T cells without regard for their “endogenous” T cell receptor repertoire, directed by antibody-defined recognition to kill malignant cells based on their expression of CEA. This is achieved by preparing chimeric molecules of hMN14 with molecules derived from T cells or related effector cell molecules, with particular enhancements, which redirect T cells or other effector cells against the tumor cells in a focused anti-tumor immune response by “re-educating” the patient's immune system.

### BRIEF DESCRIPTION OF DRAWINGS

FIG. 1 shows a chimeric antibody-T cell receptor that employs the zeta chain of the TCR. In this example, a single chain Fv (sFv) version of hMN14 is linked by a CD8α hinge to the TCR zeta chain. The CD8α hinge has been further modified to remove the cysteines involved in CD8 dimerization to improve surface expression.

FIG. 2 A shows the near absence of heterodimer molecules when the native CD8α hinge is employed, although it would be predicted to be the dominant species, with a lower net expression of chimeric molecule relative to endogenous zeta chain. FIG. 2B shows the effect of removing the cysteines, which now allows much increased net expression of chimeric molecule when heterodimer can be expressed.

FIG. 3 shows diagram and DNA sequence of chimeric hMN14 sFv IgTCR, including the CD8α hinge modified-to-remove cysteines, within a retroviral vector. The IgTCR molecule specified in this invention occupies nucleotides 2426 to 3766. (The vector sequences are incidental.)

FIG. 4 shows the DNA sequence of the VH domain (4A) and VL domain (4B) (with attached Cκ sequences) that are specific to hMN14. These sequences were modified to prepare the sFv used in FIG. 1 and FIG. 3, and similarly for other constructs.

FIG. 5 shows the effect of hMN14 IgTCR-modified T cells in killing CEA-positive tumor cells, but sparing CEA-negative cells.

FIG. 6 shows the effect of hMN14 IgTCR (signal 1) on causing sustained tumor cell killing when stimulated in conjunction with CD28 (signal 2) stimulation of the gene-modified T cells via B7 antigen expressed in the tumor cells. (A) Signal 1 alone from tumor cells leads to AICD with declining effector cell numbers, that is reversed with signal 1+2. (B) Signal 1 leads to limited duration of tumor killing because of declining T cell numbers. (C) Signal 1+2 leads to sustained tumor killing because of the sustained and expanding T cell numbers. The use of hMN14 IgCD28 to modify patient T cells will supply the second signal on contact with CEA that is necessary to suppress effector cell death and achieve sustained killing activity.

FIG. 7 shows an example of one design for hMN14 IgCD28. This also uses a modified CD8α hinge. Similar designs for other chimeric molecules with hMN14 are envisioned, with or without hinge that is the same or different.

### DETAILED DESCRIPTION OF THE INVENTION

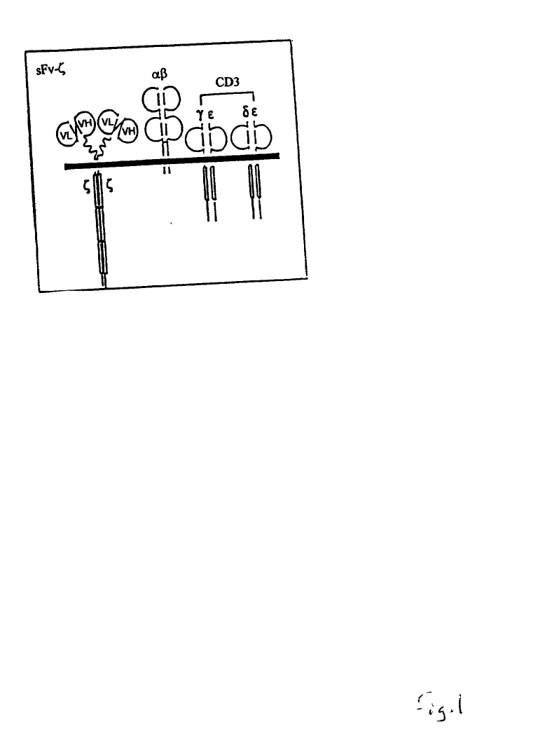

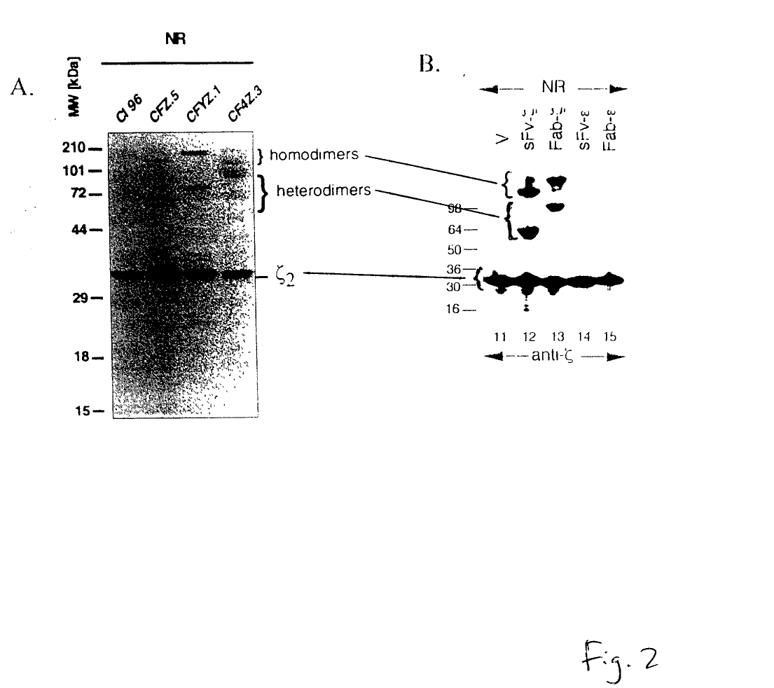

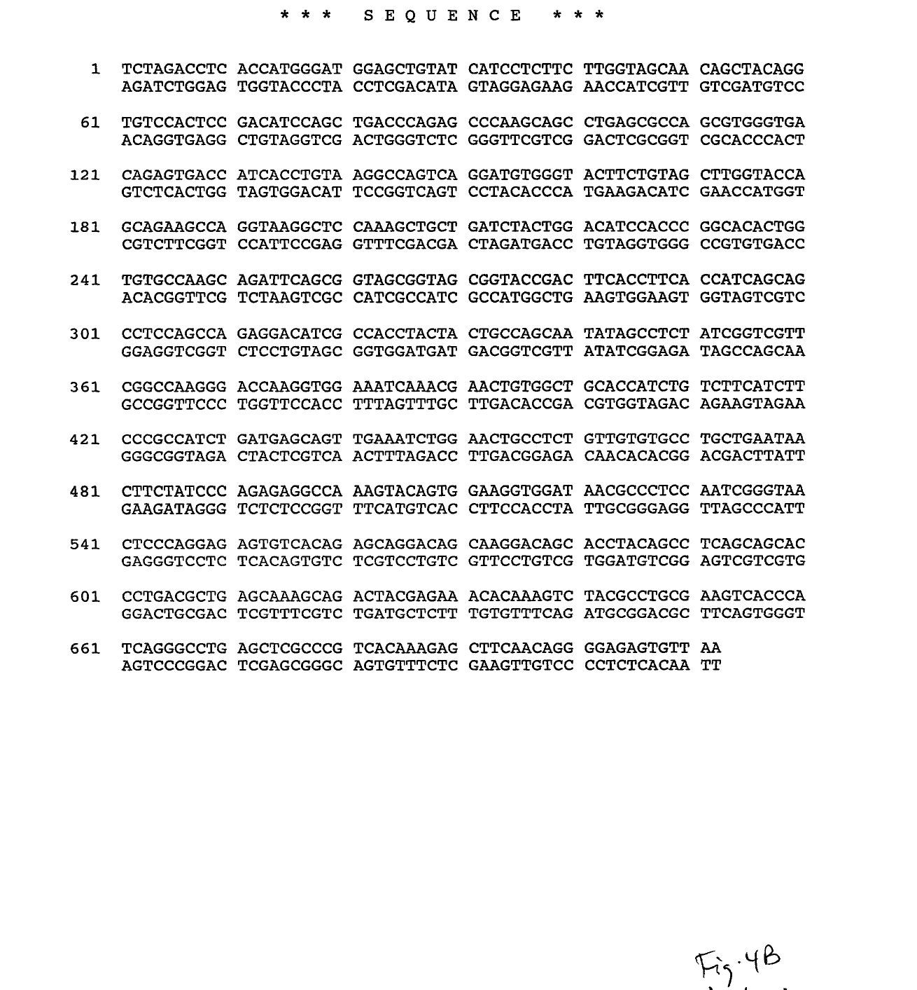

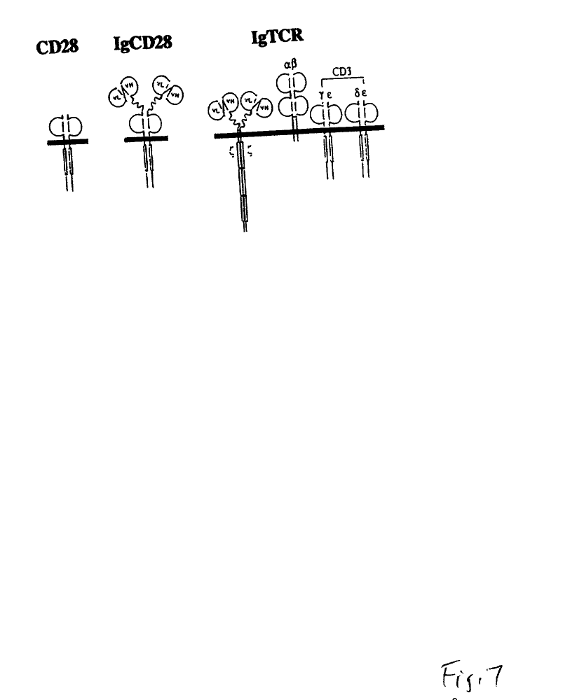

This patent is intended to cover all chimeric molecules created with the hMN14 antibody (Ig) (defined by the amino acids corresponding to the variable region sequences of FIG. 4) or its derivatives with cell surface molecules which could be used in redirecting and/or activating T cells or other effector cells in the recognition and attack against CEA-expressing tumors. The chimeric molecules of this claim includes, but is not limited to, the following molecules: IgTCR (FIG. 1), which has an antibody binding domain from the hMN14 antibody fused to one or more chains of the T cell receptor (TCR) complex; IgCD28 (FIG. 7), which has an antibody binding domain from the hMN14 antibody fused to the CD28 T cell co-receptor molecule; IgLFA-1, which has an antibody binding domain from the hMN14 antibody fused to the LFA-1 T cell co-receptor/adhesion molecule; IgCD2, which has an antibody binding domain from these specific antibodies fused to the CD2 T cell co-receptor/adhesion molecule; and by analogy, any other T cell or effector cell molecules which are usefully employed in chimeric structures with the hMN14 binding domains. The chimeric molecules may themselves incorporate cytoplasmic signaling domains, as in the foregoing examples. Or the chimeric molecules may instead be non-signaling, such as examples of Ig linked to TCR α or β chains, or Ig linked to Fc receptor (FcR) non-signaling chains, that in turn associate with signaling chains to activate cellular functions. These molecules may additionally include spacer domains or epitope tags. A single-chain Fv (sFv) version of the hMN14 has been favored for use in these constructs, but Fab or other IgG chimeric hMN14 molecules would be equally included under this invention. The initial description of some of these preparations is contained in Nolan et al, published Dec. 1, 1999. This demonstrates reduction to practice of the concepts contained herein.

The invention additionally allows for the presence of a (GSGGS)3 linker in the sFv of the Ig portion of the chimeric molecules. Whereas the sFv antibodies may frequently not fold properly to maintain stability, I included the extra serine to improve hydration and sFv folding versus the typical (GGGGS)3 linker that has been associated in some cases with abolished or diminished sFv affinity (e.g., Brinkmann et al, 1993). In the case of the hMN14 sFv, this linker led to an sFv virtually indistinguishable from the monovalent binding affinity of the parental hMN14 antibody (Nolan et al, 1999).

The invention additionally allows for the modification-to-remove cysteines in the CD8α hinge domain to improve the surface expression of the chimeric molecules (Nolan et al, 1999). Free cysteines of the hinge of the heterodimer of zeta: sFv-hinge-zeta target this molecular complex for destruction, reducing the net amount of chimeric molecule expression on the cell surface. (The homodimer (sFv-hinge-zeta)₂ has safe pairing of cysteines to spare this specific configuration from destruction. More heterodimer is expected because of binomial considerations where the endogenous zeta exceeds the transduced zeta chimera as is typical.) This principle is demonstrated by the poor expression of heterodimers of such molecules where the cysteine residues are retained (Moritz et al, 1995) and their excellent expression when I modified-to-remove these cysteines (Nolan et al, 1999) (FIG. 2). The efficacy of T cell functions through surface receptors are generally higher with higher surface expression, which the rescue (i.e., non-destruction) of heterodimers would allow. These chimeric molecules are introduced into patient T cells by gene therapy techniques, such as by retroviral vector transduction or other methods. This method of improving cell surface expression is cross-referenced (Junghans Provisional Patent No. 60/250,087).

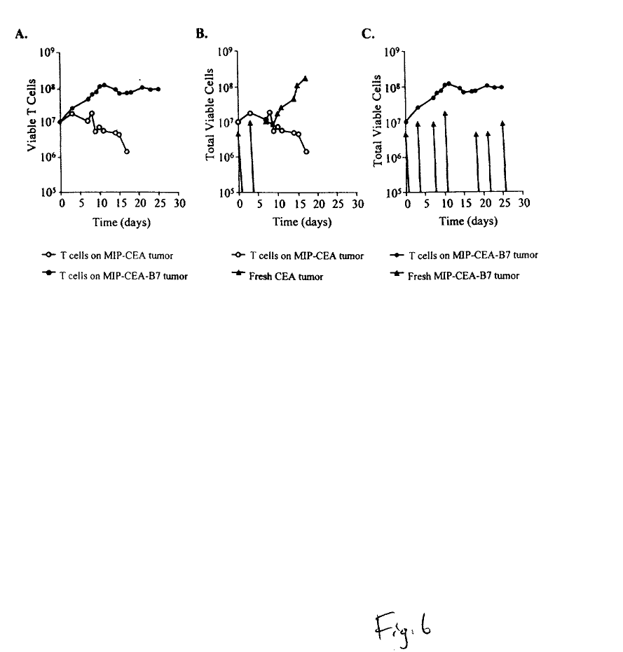

In one example, IgTCR (FIG. 1) provides signal 1, which directs T cell killing; IgCD28 (FIG. 7) provides signal 2, which suppresses activation induced cell death of T cells and allows sustained proliferation and survival; and IgLFA1, which provides signal 3 and supports secretion of interleukin 2, an essential T cell growth factor. Combinations of signals can yield improved T cell survival and tumor cell killing (FIG. 6). The invention allows for use of these and/or analogous chimeric molecules of hMN14 alone or in any combination.

The combination use of such chimeric molecules in treatment of cancers is a further part of the claim. This applies an understanding that more than one signal is required for sustained antitumor efficacy. This application specifically envisions that the same antibody binding domain is applied in the additional chimeric receptor molecules such that encounter with the same tumor antigen successfully triggers more than one signal in the effector cell. Alternatively, additional signaling chimeric molecules may have engineered Ig specificities which direct them to different surface molecules on the tumor cell, rather than to the same one, to avoid binding site competition or to regulate the amount of receptor stimulation where this regulation enhances the desired outcome of antitumor efficacy in therapy.

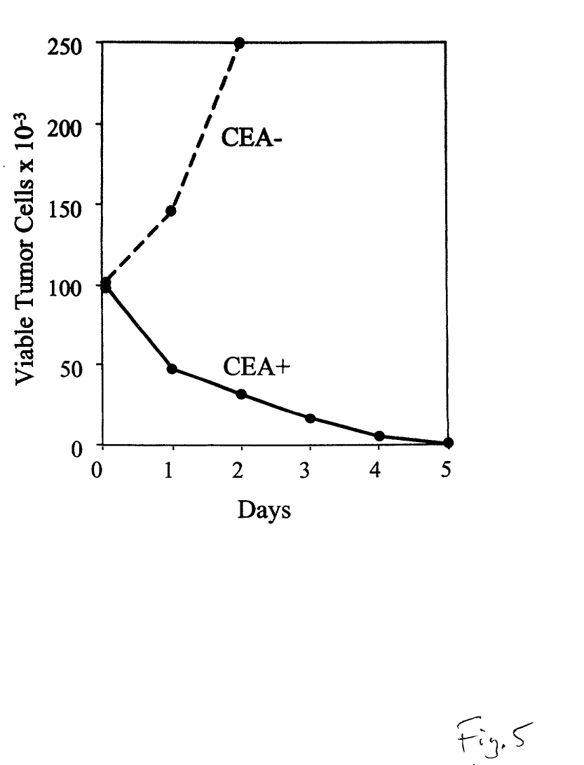

The purpose is to educate immune effector cells to attack CEA-expressing tumor cells. Advantages are that the sequences used to recognize CEA in hMN14 are humanized and of high affinity, their conjugation with T cell molecules leads to direct recognition of CEA+ tumors by human T cells with proven efficacy, and hinge and sFv linker modifications make the Ig folding and surface expression more efficient with advantages in anti-tumor activity. Presently, treatments for CEA+ cancers are surgery, chemotherapy and radiation, none of which is curative for metastatic disease. A critical component of this therapy is the specific antibody, hMN14, that recognizes the CEA. No other IgTCR or Ig-T cell molecules has the amino acid sequence of the hMN14 CEA-recognition domain which I employ, and which I have proven to be effective (FIG. 5). There is no patent of the hMN14 sequence in chimeric state with T cell or other effector cell molecules, or with the use of a modified hinge structure.

An invention exists as to the general chimeric Ig molecules with cell receptor proteins (Capon et al, 1996). My invention is distinguished by targeting the CEA antigen, by the uniqueness of the hMN14 antibody sequences employed, by the new concept in modification of hinge and sFv linker domains that improves the cell surface expression, and by the combination of such anti-CEA hMN14 receptor molecules expressed in effector cells which are stimulated in concert specifically by the same tumor antigen or by a different tumor antigen or antigens. These chimeric hMN14 molecules are in addition to and outside of the claims of the hMN14 patent (Hansen et al., 1994), but reference the uniqueness of these sequences (FIG. 4).

what is claimed is:

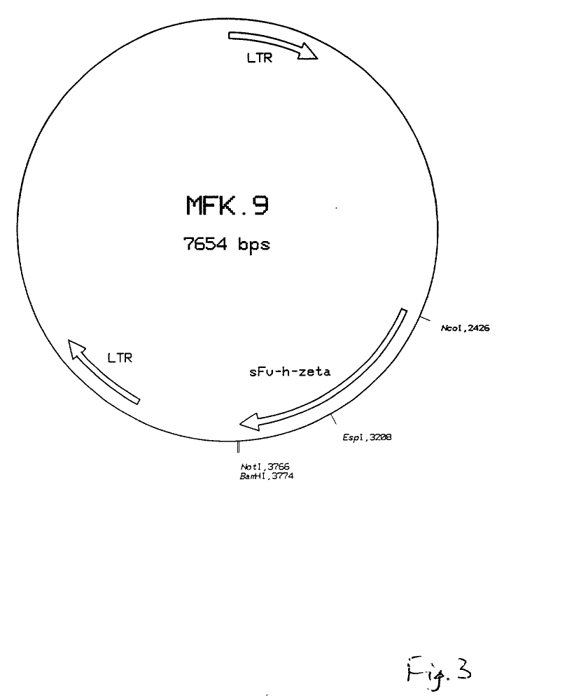

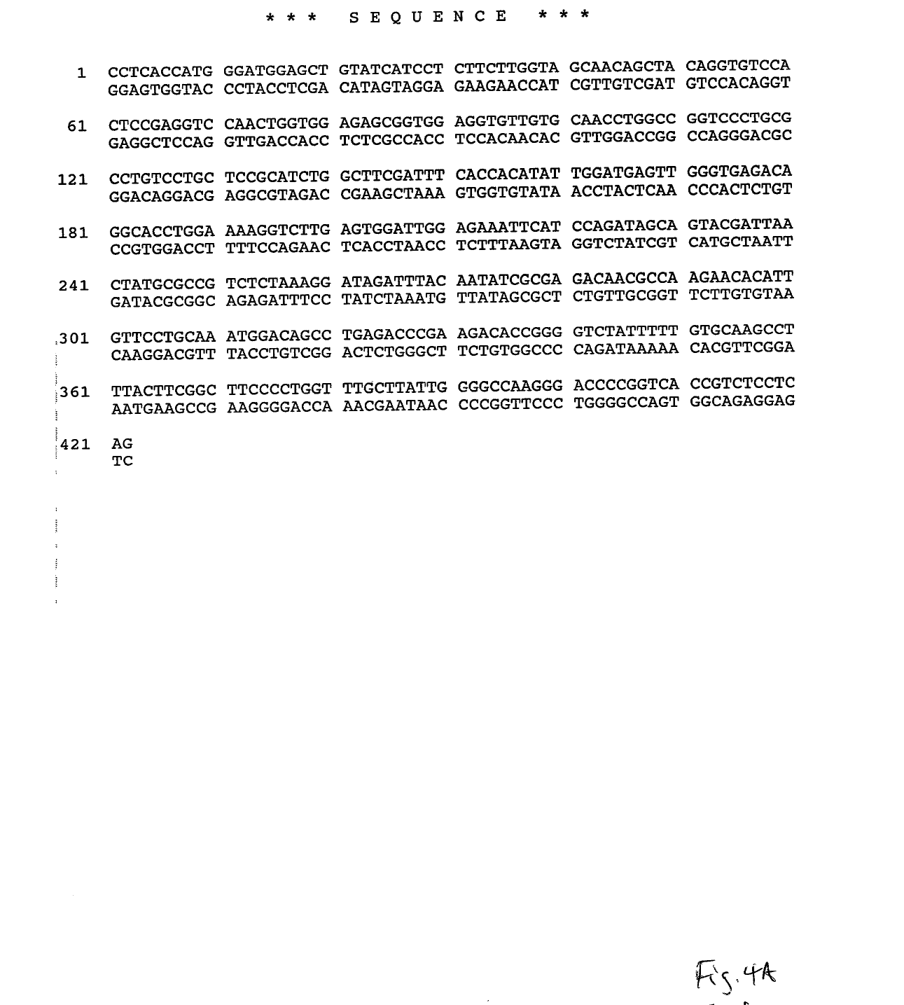

1. A chimeric molecule comprised of the CEA binding domain of humanized antibody MN14 as a single chain antibody with a (GGSGS)3 linker, the zeta signaling chain of the T cell receptor and an intervening CD8α hinge in which the cysteine residues have been mutated, with the sequence of FIG. 3.

2. A molecule of claim 1 in which other signaling chains of T cells or other cell types are substituted, or in which a different hinge molecule or no hinge molecule is substituted, or a combination thereof.

3. A molecule of claim 1 or 2 in which at least one of the CDRs of the heavy chain of MN14 and one of the CDRs of the light chain of MN14 are preserved in a form (e.g., sFv or Fab) that maintains the binding of the CEA antigen, and/or in which the linker is of different composition.

4. A molecule of claim 1, 2 or 3 which has been modified in DNA or protein sequence but which retains the specificity and action of these molecules.

5. The use of molecules of claims 1, 2, 3 or 4 expressed in T cells or NK cells or other effector cells to treat patients with cancers expressing the CEA antigen.

6. The combination use of molecules of claims 1, 2, 3 or 4 expressed in T cells or NK cells or other effector cells to treat patients with cancers expressing the CEA antigen, together with each other or with heterologous constructs to engage additional stimulatory and functional properties of the effector cells to enhance the antitumor therapeutic efficacy.


SEQUENCE LISTING (6 sequences)

SEQ ID NO: 1 - DNA, 7654 bp, Homo sapiens
  CDS (2428)..(3759): Chimeric IgTCR sequence contained in retroviral vector. Retroviral vector sequence (non-coding regions) are incidental to the invention. The translated (coding region) is relevant to the invention. (pertinent to Figure 3.)
aagcttgcatgcctgcaggtcgactctaggcacataaagaaaaacataactaaccaagctgcagccgagacagtgaaaagaaccgttaaaacggtttgttttaaataaactgaattatttagagtcatttctttggtaggaaagtacattggcacgtaaaggagcccaaagcaatctgtggaaagcccaggctgggagcccagcagtttgcatcccctcctggcgtgtacctaagggtttcttaattgtgtggtttctaaatcttccagagggtttgtctcattcacttccacttcggtgcacaatacttggacgcggatttactgtcttagcatctatcggtggcccttcgattgaggctgaacctgaggcccacttcttcagcttgttaaggagagcacaagcaccagaagaggctgacccggcagacctgtgggcatttttaacaagggcctcctgggtctgtgggaggcaggcttacataaggtgcaaattagaaatataaataataagcccatatcaatttgtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggcttctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaggtggctcaggatcgggtggatccggctctggtggctcaggatcggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccagtgctaagcccaccacgacgccagcgccgcgaccaccaacaccggcgcccaccatcgcgtcgcagcccctgtccctgcgcccagaggcggctcggccagcggcggggggcgcagtgcacacgagggggctggacttcgccctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaactcgacgcggccgcggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctctctgagtgattgactacccgtcagcggggtctttcagtttctcccacctacacaggtctcactaacattcctgatgtgccgcagggactccgtcagcccggtttttgtttataataaaatgcaagaacagtgttcccttcaagccagactacatcctgactctcggctttataaaagaatgttgaagggctctgtggactatctgccacacgactttttaagatttttatgcctcctggatgagggatttagtcaatctatcctcgtctattttgctggcttctccgtattttaaatttctagtttgcactcccttcctgagagcacggcgattgcagagtagttaatactctgagggcaggcttctgtgaaaaggttgcctgggctcagtgtgagattttgccataaaaaggggtcctgcccctgtgtacagacagatcggaatctagagtgcatactcagagtccccgcggttccggggctctgatctcagggcatctttgcctagagatcctctacgccggacgcatcgtggccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggctcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 2 - protein, 443 aa, Homo sapiens
MGWSCIILFLVATATGVHSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFALDPKLCYLLDGILFIYGVILTALFLRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 422 bp, Homo sapiens and Mus sp.
  CDS (8)..(421): hMn14 VH, humanized (CDR-grafted) anti-CEA antibody heavy chain V region (aa20-138) with leader (aa1-19) (pertinent to Fig. 4A.)
cctcaccatgggatggagctgtatcatcctcttcttggtagcaacagctacaggtgtccactccgaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctcctcag

SEQ ID NO: 4 - protein, 138 aa, Homo sapiens and Mus sp.
MGWSCIILFLVATATGVHSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 5 - DNA, 712 bp, Homo sapiens and Mus sp.
  CDS (14)..(712): hMN14 VLCK, humanized (CDR-grafted) anti-CEA antibody kappa light chain V region (aa20-126), with human constant CK domain (aa127- 232) and leader (aa1-19). (pertinent to Figure 4B.)
tctagacctcaccatgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 6 - protein, 232 aa, Homo sapiens and Mus sp.
MGWSCIILFLVATATGVHSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC